(19) United States
(12) Patent Application Publication
(10) Pub. No.: US 2008/0176913 A1
(43) Pub. Date: Jul. 24, 2008

(54) TRANSDERMAL COMPOSITIONS OF PRAMIPEXOLE HAVING ENHANCED PERMEATION PROPERTIES

(76) Inventors: Arnaud Grenier, Steinbrunn le Haut (FR); Dario Norberto R. Carrara, Oberwil (CH)

Correspondence Address: WINSTON & STRAWN LLP;PATENT DEPARTMENT, 1700 K STREET, N.W., WASHINGTON, DC, 20006, US

(21) Appl. No.: 11/770,194

(22) Filed: Jun. 28, 2007

Related U.S. Application Data
(60) Provisional application No. 60/818,089, filed on Jun. 29, 2006.

(51) Int. Cl. A61K 31/4425; A61P 25/14

(52) U.S. Cl. 514/367

(57) ABSTRACT

A pharmaceutical composition for transdermal or transmucosal delivery of an active agent to treat a movement disorder such as Parkinson's disease. The composition provides enhanced transdermal or transmucosal delivery of the active agent by including an alkanolamine as a permeation enhancer with a carrier of water and at least one short-chain alcohol and with the composition having a neutral pH. The composition provides controlled and sustained release of the active agent suitable for daily administration.

This application claims the benefit of provisional application 60/818,089 filed Jun. 29, 2006, the entire content of which is expressly incorporated herein by reference thereto.

### FIELD OF INVENTION

The invention relates generally to transdermal drug delivery, and more particularly to transdermal compositions and methods of administering an active agent such as pramipexole. The invention additionally relates to a non-occlusive transdermal semi-solid composition containing pramipexole, which is chemically stable and which provides enhanced permeation of the drug through the skin or the mucosa.

### BACKGROUND OF THE INVENTION

Parkinson's disease (PD) is a hypokinetic disorder comprised of four features: (i) bradykinesia (slowness and poverty of movement); (ii) muscular rigidity (an increase in the resistance of the muscles to passive movement); (iii) resting tremor; and (iv) abnormalities of posture and gait. In Parkinson's disease, the dopaminergic system is deficient due to the degeneration of dopaminergic neurones in the nigrostriatal pathway, which allows the cholinergic system to hold unopposed sway, resulting in abnormal control of muscular activity. Thus, the two main approaches to treating Parkinson's disease have been replenishment of the stores of dopamine and reduction of excessive cholinergic action by acetylcholine antagonists. While it is difficult to estimate the number of people affected by this disease, because the symptoms of the disease are often mistaken for the normal results of aging or are attributed to other diseases, Parkinson's disease occurs in people all over the world, in all ages.

Presently, the most effective anti-Parkinson drug available is levodopa. When levodopa is taken alone, the body breaks down about 95% of the drug into dopamine before it reaches the brain, producing a lot of side effects. Combining levodopa with another drug such as carbidopa (e.g., SINEMET® of Merck) or benserazide enables more levodopa to enter the brain before it converts into dopamine. As many as half the people who take this drug for two to five years begin to notice fluctuations in the drug's effectiveness, known as an on-off effect. Others develop dyskinesia—involuntary movements such as jerking or twitching. As Parkinson's disease progresses, the effectiveness of the combination also decreases and patients require higher and more frequent doses to control their symptoms.

As an alternative to levodopa, dopamine agonists have played an important role in treating Parkinson's disease. Dopamine agonists, such as pergolide, lisuride and pramipexole, mimic the action of dopamine by activating nerve cells in the striatum. Dopamine agonists are increasingly used alone in the early stages of Parkinson's disease in order to lower a patient's risk of developing the dyskinesia associated with levodopa therapy. Later in the course of the disease they are more likely to be combined with carbidopa or levodopa to alleviate that drug's on-off effects. All available dopamine agonists stimulate D2 receptors, which is believed to be clinically beneficial.

One of the dopamine agonists indicated for the treatment of idiopathic Parkinson's disease is pramipexole, which has become one of the most widely used dopamine agonists because of its proven efficacy. Pramipexole is presently marketed as the hydrochloride salt in an immediate-release tablet for the treatment of Parkinson's Disease (MIRAPEX® of Boehringer Ingelheim). Pramipexole dihydrochloride has the chemical name (S)-2-amino-4,5,6,7-tetrahydro-6-(propylamino)benzothiazole dihydrochloride monohydrate and a structural formula as shown below:

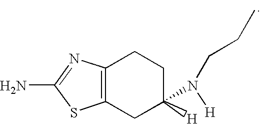

Pramipexole, an indolone compound, is a nonergot dopamine agonist with a high relative in vitro specificity and full intrinsic activity at the D2 subfamily of dopamine receptors, and binds to D3 receptors with higher affinity than to D2 or D4 receptor subtypes. While the precise mechanism of action of pramipexole as a treatment for Parkinson's disease is unknown, it is believed that pramipexole provides treatment by stimulating dopamine receptors in the striatum. This conclusion is supported by electrophysiological studies in animals which demonstrate that pramipexole influences striatal neuronal firing rates via activation of dopamine receptors in the striatum and the substantia nigra, the site of neurons that send projections to the striatum. Pramipexole, its chemical structure, processes for its preparation and therapeutic uses thereof are more fully described in U.S. Pat. Nos. 4,452,808, 4,824,860, and 6,770,761.

Administration of any active pharmaceutical agent, including pramipexole and other anti-Parkinson agents, should preferably be provided by an administration regime—the route of administration and the dose regimen—that is as simple and non-invasive as possible in order to maintain a high level of compliance by the patient. Oral administration is an administration regime that is commonly used because it is relatively simple to follow, but oral administration may cause many side effects and complications, including, among others, complications associated with gastrointestinal irritation and drug metabolism in the liver. For instance, oral administration of pramipexole can cause serious adverse effects such as nausea, dizziness, drowsiness, somnolence, insomnia, constipation, unusual weakness, stomach upset and pain, headache, dry mouth, hallucinations, difficulty moving or walking, difficulty breathing, confusion, restlessness, leg or foot swelling, fainting, twitching, chest pain, unusually fast or slow heartbeat, muscle pain, vision problems, fever, severe muscle stiffness, and sudden irresistible urge to sleep. Even administration of small amounts of pramipexole, which is typically administered at a daily does of about 1.5 to 4.5 mg, with bioavailability of 90%, is associated with considerable side effects. An alternative route of administration is therefore desired.

Recently, administration of active pharmaceutical agents through the skin—the “transdermal drug delivery”—has received increased attention because it provides not only a simple dosage regime but also a relatively slow and controlled release of an active agent into the system, ensuring a safe and effective administration of the active agent. Advantageously, transdermal administration can totally or partially alleviate the side effects associated with oral administration. For example, U.S. Pat. No. 5,112,842 explains that continuous transdermal delivery of pramipexole provides a number of advantages, such as sustained pramipexole blood levels, which is believed to provide a better overall side effect profile than typically associated with oral administration; absence of first-pass effect; substantial avoidance of gastrointestinal and other side effects; and improved patient acceptance.

Transdermal administration of pramipexole by means of a patch, also known as transdermal therapeutic system (TTS), is known. For example, U.S. Patent Application Publication No. US 2004/0253299 discloses a reservoir-TTS containing pramipexole or a pharmaceutically acceptable salt or derivative thereof, and a chelate former or an antioxidant as a stabilizer as applicable, which is stable to decomposition and provides for release of the active ingredient over a period of three or more days. U.S. Patent Application Publication No. US 2006/0078604 discloses a transdermal drug delivery system for topical application of pramipexole, contained in one or more polymeric and/or adhesive carrier layers proximate to a non-drug containing polymeric backing layer, where the delivery rate and profile is controlled by adjusting the moisture vapor transmission rate of the polymeric backing layer. U.S. Pat. No. 6,221,383 discloses a TTS comprising a blend of polymers, which provides a pressure-sensitive adhesive composition for transdermal delivery of drugs.

Transdermal therapeutic systems or patches, however, present many drawbacks, such as skin irritation caused by high drug loading per cm², adhesives used in the patch, and the occlusive nature of the patch. Therefore, a non-patch, non-occlusive composition for transdermal delivery of an anti-Parkinson agent is desired.

Certain non-patch, transdermal compositions containing pramipexole are known. U.S. Pat. No. 6,383,471 discloses a pharmaceutical composition, which comprises (a) a hydrophobic therapeutic agent having at least one ionizable basic functional group and (b) a carrier comprising (i) a pharmaceutically acceptable inorganic or organic acid; (ii) a surfactant selected from the group consisting of non-ionic hydrophilic surfactants having an HLB value greater than or equal to about 10, ionic hydrophilic surfactants, hydrophobic surfactants having an HLB value less than 10, and mixtures thereof; (iii) optionally a triglyceride; and (iv) optionally a solubilizer. U.S. Pat. No. 6,833,478 discloses a method for increasing the solubility of an anti-Parkinson agent in a lipophilic medium, the method comprising admixing the agent with a solubility-enhancing amount of an N,N-dinitramide salt, wherein ionization of the agent results in a biologically active cationic species in association with an anionic counter-ion. Pramipexole is not included as one of the anti-Parkinson agents disclosed in this publication. U.S. Pat. No. 6,929,801 discloses a transdermal drug delivery system comprising a therapeutically effective amount of an anti-Parkinson agent such as pramipexole, at least one dermal penetration enhancer which is a skin-tolerant ester sunscreen, and at least one volatile liquid.

Despite these disclosures, it would be further advantageous to provide a transdermal composition of pramipexole which provides sustained release of pramipexole such that the composition can be administered less frequently, for example, once a day. Conventionally, pramipexole is administered several times a day. Hubble et al., Clinical Neuropharmacology 18(4), 338-347 (1995) describes administration of pramipexole three times a day in patients with early Parkinson's disease. Steady-state pharmacokinetic properties of pramipexole, when administered three times a day in the form of pramipexole dihydrochloride tablets as reported in Wright et al., Journal of Clinical Pharmacology 37, 520-525 (1997), concludes that steady-state pharmacokinetic characteristics are linear up to a daily dose of 4.5 mg with such multiple administrations.

U.S. Patent Application Publication No. US 2006/0110454 states that the prior art recognizes reduced side effect profile of once daily dosage form, compared to thrice daily immediate release dosage form. U.S. Patent Application Publication No. US 2005/0226926 also discloses that a three times daily dosing regimen for immediate-release pramipexole dihydrochloride tablets is well tolerated, but that patient compliance would be much improved if a once-daily regimen were possible. Because Parkinson's disease is an affliction that becomes more prevalent with advancing age, a once-daily regimen is noted as especially useful in enhancing compliance among elderly patients. Thus, a once daily administration of an anti-Parkinson agent such as pramipexole would be desirable. Such a composition would simplify the administration regime of the drug by reducing the number of daily application and improve patient compliance, while also reducing adverse events and side effects associated with an immediate release formulation, such as high plasma peaks.

In addition, it would be advantageous to provide a transdermal composition of an anti-Parkinson agent which allows improved permeation of the agent while maintaining the stability of the agent in the composition. Although transdermal formulations are generally known, it can be difficult to find a permeation enhancer that is compatible and effective with a particular drug, considering that even structurally related permeation enhancers can provide completely different permeation profiles when used in combination with a drug. These effects have been studied with triethanolamine.

For instance, Gwak H S, Choi J S and Choi H K., “Enhanced bioavailability of piroxicam via salt formation with ethanolamines,” Int'l J. Pharm. 297(1-2): 156-61 (June 2005) found that formation of piroxicam triethanolamine salts, while increasing bioavailability of the drug piroxicam, decreased skin permeability of the drug. This article concluded that piroxicam salt formation with MEA and DEA improved the physicochemical properties and enhanced the skin permeability of piroxicam, while the solubility and permeation rate of piroxicam triethanolamine salt was lower than those of piroxicam in most of vehicles tested. Cheong H A and Choi H K, “Effect of ethanolamine salts and enhancers on the percutaneous absorption of piroxicam from a pressure sensitive adhesive matrix,” Eur. J. Pharm. Sci. 18(2):149-53 (February 2003) also investigated the effects of piroxicam-ethanolamine (PX-EA) salts formation on the percutaneous absorption of piroxicam through hairless mouse skin from a pressure sensitive adhesive (PSA) matrix, and found the permeation rates of piroxicam and PX-EA salts from the PSA matrix to be highest for piroxicam-monoethanolamine salt, followed by piroxicam-diethanolamine salt, piroxicam, and then piroxicam-triethanolamine salt. Similarly, Gwak H S and Chun I K., “Effect of vehicles and penetration enhancers on the in vitro percutaneous absorption of tenoxicam through hairless mouse skin,” Int'l J. Pharm. 236(1-2):57-64 (April 2002) reported that triethanolamine did not show a significant enhancing effect on in vitro permeation of tenoxicam but rather decreased the fluxes of tenoxicam when added to propylene glycol with fatty acids, in contrast to tromethamine, which showed an enhancing effect on the in vitro permeation of tenoxicam from saturated solutions through dorsal hairless mouse skin by the increased solubility. The preparation of alkanolamine, including monoethanolamine, diethanolamine, triethanolamine and propanolamine, complexes of non-steroidal anti-inflammatory mefenamic acid (MH) has also been studied as an attempt to increase the transdermal flux of MH in Fang L, Numajiri S, Kobayashi D and Morimoto Y, “The use of complexation with alkanolamines to facilitate skin permeation of mefenamic acid,” Int'l J. Pharm. 262(1-2):13-22 (August 2003). A marked enhancement of MH flux from the alkanolamine complexes through hairless rat skin membrane was observed only in the presence of a lipophilic enhancer system consisting of isopropyl myristate and ethanol in a 9 to 1 ratio. Among the alkanolamines examined, the propanolamine complex had the greatest enhancing effect on the permeation of MH. Another study, Fang L, Kobayashi Y, Numajiri S, Kobayashi D, Sugibayashi K and Morimoto Y, “The enhancing effect of a triethanolamine-ethanol-isopropyl myristate mixed system on the skin permeation of acidic drugs,” Biol. Pharm. Bull. 25(10):1339-44 (October 2002), investigated the effects of a ternary enhancer system consisting of triethanolamine (T), ethanol (E) and isopropyl myristate (I) on the in vitro skin permeation of acidic, basic and neutral drugs using excised hairless rat skin. The EI binary enhancer system produced marked improvement in penetration of all of the tested drugs. However, addition of triethanolamine to the EI system resulted in a greater enhancing effect only for acidic drugs with a carboxyl group. Further, while mefenamic acid exhibited the highest enhancing effect of all the acidic drugs tested, substitution of triethanolamine in the TEI enhancer system with another amine resulted in even greater flux of mefenamic acid, approximating 14-180 times greater flux. It was also found that the transdermal flux of mefenamic acid increased by increasing the triethanolamine concentration in the TEI system. Using differential scanning calorimetry, Fourier transform infrared spectroscopy, and X-ray crystallographic studies, this study further demonstrated that mefenamic acid and each alkanolamine tested (propanolamine, diethanolamine, triethanolamine) formed an ion pair complex. The amine complexes had a lower melting point and higher solubility in water compared with pure mefenamic acid (see Fang L, Numajiri S, Kobayashi D, Ueda H, Nakayama K, Miyamae H, and Morimoto Y, “Physicochemical and crystallographic characterization of mefenamic acid complexes with alkanolamines,” J. Pharm. Sci. 93(1):144-54 (January 2004)).

Use of triethanolamine in a transdermal product of an anti-inflammatory drug has also been studied. U.S. Pat. No. 4,533,546 discloses hydro-alcoholic compositions comprising a phenylacetic acid-type anti-inflammatory analgesic agent and having a pH in the range of from 7.0 to 9.0. This document requires use of water-soluble organic amine, of which mono-, di- and tri-(lower alkanol)amines are preferred, with diisopropanolamine being especially preferred, in an amount much greater than what is required to neutralize the carboxyvinyl polymer. The amine is used in an amount such that the final gelled ointment has a pH in the range of 7.0 to 9.0, preferably 7.0 to 8.0, and more preferably 7.3 to 7.8. U.S. Pat. No. 5,814,659 discloses compositions comprising a topical analgesic agent, an alcohol, a chaotropic agent, and an unsaturated fatty acid, wherein the pH is adjusted to about 7.5 to 8.0 by adding a pharmaceutically acceptable organic base, e.g., triethanolamine, to ensure stability of the gel. U.S. Pat. No. 5,916,587 discloses transdermal delivery matrix comprising piroxicam; an adhesive polymer; an absorption assistant selected from the group consisting of dimethylsulfoxide, dimethylacetamide, dimethylformamide, alkanolamine, alkylamine, diethyleneglycol monoethylether, and N-alkyl pyrrolidone; and a penetration enhancer selected from the group consisting of alkylene glycol, propylene glycol, [1-alkylazacycloheptane-2-one, 1-dodecylazacycloheptane-2-one,] lauric diethanolamide, oleic acid, and a polyethylene glycol.

Additionally, U.S. Pat. No. 6,855,702 discloses combretastatin A-4 (an anti cancer drug) phosphate prodrug salts that have increased in vivo solubility relative to the solubility of native combretastatin A-4, readily regenerate combretastatin A-4 under physiological conditions, and, during regeneration, produce physiologically tolerable organic amines, or physiologically tolerable amino acids or amino acid esters that are readily metabolized in vivo. Salts are formed from substituted aliphatic organic amines, such as ethanolamine, diethanolamine, ethylenediamine, diethylamine, triethanolamine, tromethamine, glucamine, N-methylglucamine, ethylenediamine, 2-(4-imidazolyl)ethyl amine, choline, hydrabamine and stereoisomers thereof. U.S. Pat. No. 6,217,852 discloses a transdermal device and a method for reducing or eliminating irritation or sensitization caused by an irritating or sensitizing non-zwitterionic drug when it is delivered transdermally while achieving therapeutically effective transdermal fluxes. In a preferred embodiment, the transdermal device comprises a reservoir and a backing, wherein the reservoir contains a conjugated or non-conjugated weak acid or base to control the pH within 3 to 6 pH units below the pKa of the drug. Preferred drug is selected from fluoxetine, paroxetine, citalopram, olanzapine, raloxifen, fentanyl, chlorpromazine, and oxybutynin. U.S. Pat. No. 5,498,417 discloses a transdermal patch for delivering an indirect-acting phenyl propanolamine drug through human skin, the patch comprising a silicone-coated release layer, an adhesive/drug mixture coating on the release layer, a carrier film laminated to the coated release layer, a propylene glycol permeation enhancer, and a triethanolamine pH control additive which also acts as a permeation enhancer. U.S. Pat. No. 5,643,584 discloses compositions for topical administration of tretinoin to the skin comprising sufficient base to attain a pH in the range of from 4.0 to 7.0, wherein the base is sodium hydroxide or triethanolamine.

Thus, to improve upon prior art formulations, what is needed is a transdermal composition of an anti-Parkinson agent having enhanced permeation profiles, which can be provided in a non-patch or non-occlusive form and which can provide a sustained release of the anti-Parkinson agent.

### SUMMARY OF THE INVENTION

The invention relates to a transdermal composition or a dosage form comprising a therapeutically effective amount of pramipexole or a pharmaceutically acceptable salt thereof, a carrier comprising a mixture of water and at least one short-chain alcohol, and at least one permeation enhancer comprising an alkanolamine in an amount sufficient to increase permeation through dermal or mucosal surfaces compared to formulations where the alkanolamine is not utilized. The apparent pH of the dosage form is generally between about 7 and 9.

Pramipexole can be provided as a free base or as a salt, such as a hydrochloride or dihydrochloride salt. In an example, pramipexole is present at a concentration of about 0.5 to about 5 weight percent, or about 1 to 5 weight percent of the composition, calculated as free base equivalent.

The alkanolamine can be selected from monoethanolamine, diethanolamine, triethanolamine, diisopropylamine, meglumine, and derivatives and mixtures thereof. Preferably, the alkanolamine is present in an amount of about 15 to 40% by weight of the dosage form and is triethanolamine. Advantageously, the dosage form exhibits transdermal flux of pramipexole that is greater than that of a composition that does not contain alkanolamine but contains an inorganic pH-adjusting agent.

For the carrier, the short-chain alcohol can be ethanol, propanol, isopropanol, and mixtures thereof. The alcohol is typically present in an amount of about 27 to 54% by weight of the dosage form. The carrier can further comprise any additional conventional pharmaceutical excipients as suitable and desired, including one or all of a non-volatile solvent, an antioxidant, a thickening agent, or a secondary permeation enhancer.

The dosage form can be produced by mixing the ingredients into a homogenous composition, and can be provided in any desired dosage, for example as a unit dosage or a multi-dosage in an appropriate container.

The dosage form can be administered by applying on an area of skin of the subject in an amount sufficient to provide a therapeutic concentration of pramipexole in the bloodstream of the subject. Thus, the dosage form can be used to treat various movement disorders, such as Parkinson's Disease, Restless Legs Syndrome, Tourette's Syndrome, Chronic Tic Disorder, Essential Tremor, and Attention Deficit Hyperactivity Disorder. The dosage form can be administered in any desired amount and frequency, such as in an amount up to about 10 grams per day where the dosage form contains about 0.5 to 5 weight percent of pramipexole. The surface area of skin for receiving the dosage form can also vary as desired, with the preferred area being about 50 to about 1000 cm², or about 100 to about 400 cm².

In an embodiment, the dosage form provides a sustained, steady-state delivery of pramipexole for an extended time, for example for about 24 hours.

### BRIEF DESCRIPTION OF THE DRAWINGS

The features of the invention will be further described in the following detailed description and accompanying drawings in which:

FIGS. 1A-2B graphically illustrate the effect of pH on the cumulative drug permeation and drug instant flux of pramipexole hydrochloride;

FIGS. 3A-3B graphically illustrate the effect of the pH-adjusting agent on the cumulative drug permeation and drug instant flux of pramipexole hydrochloride;

FIGS. 4A-4B graphically illustrate the effect triethanolamine concentration on the cumulative drug permeation and drug instant flux of pramipexole hydrochloride; and

FIGS. 5A-5B graphically illustrate the effect of triethanolamine on the cumulative drug permeation and drug instant flux of pramipexole hydrochloride of pramipexole salt versus pramipexole free base.

FIGS. 6A-6B graphically illustrate the effect of the pH-adjusting agent on the cumulative drug permeation and drug instant flux of nicotine.

FIGS. 7A-7B graphically illustrate the effect of the pH-adjusting agent on the cumulative drug permeation and drug instant flux of pramipexole hydrochloride;

FIGS. 8A-8B graphically illustrate the effect of the pH-adjusting agent on the cumulative drug permeation and drug instant flux of pramipexole hydrochloride;

### DETAILED DESCRIPTION OF PREFERRED EMBODIMENTS

The invention relates to a transdermal composition containing an anti-Parkinson agent, such as pramipexole or a pharmaceutically acceptable salt thereof. The invention also relates to a pharmaceutical composition of an anti-Parkinson agent, wherein the composition provides continuous and sustained release of the active agent over an extended period of time.

As used herein, the term “anti-Parkinson agent” or “anti-Parkinson drug” is understood to include any drug or active agent that is effective to treat Parkinson's disease or other such neurological or movement disorders having symptoms similar to those of Parkinson's disease, e.g., Restless Leg Syndrome, Tourette's Syndrome, Chronic Tic Disorder, Essential Tremor, and Attention Deficit Hyperactivity Disorder. Preferably, the anti-Parkinson agent is a dopamine agonist such as pramipexole or other indolone compounds.

As used herein, the term “transdermal” is understood to also include both transdermal and transmucosal delivery of an active agent.

Pramipexole, its chemical structure, processes for its preparation and therapeutic uses thereof are more fully described in U.S. Pat. Nos. 4,452,808, 4,824,860, and 6,770,761, the contents of each of which are expressly incorporated herein by reference. As used herein, the term “pramipexole” includes pharmaceutically acceptable salts thereof. The skilled artisan is well aware of the different types of pharmaceutically acceptable salts that can be selected for formulation and use in the composition. Preferably, pramipexole is used in the form of its hydrochloride salt.

As used herein, the amount or concentration of pramipexole is expressed as measured by the free base equivalent of pramipexole.

In one embodiment, the invention provides a non-occlusive, non-patch form of transdermal composition containing an anti-Parkinson agent such as pramipexole in a carrier comprising water and a short-chain alcohol, such as ethanol, propanol, and isopropanol. The composition can additionally contain other conventional pharmaceutically acceptable excipients as desired, including non-volatile solvents, antioxidants, and thickening agents. The composition can further comprise a permeation enhancer, such as an alkanolamine, e.g., monoethanolamine, diethanolamine, triethanolamine, diisopropylamine, and meglumine. An alkanolamine permeation enhancer can be used in combination with another permeation enhancer.

The composition can be provided in any dosage form suitable for topical application, including a lotion, a cream, a gel, a solution, or a patch, although a non-occlusive form is preferred to eliminate the disadvantages associated with an occlusive form such as patch.

The anti-Parkinson agent can be provided in any desired amount in the present composition. For example, the agent can be provided in an amount of about 0.5 to 5% by weight of the composition, measured by the pramipexole free base equivalent. The total amount of administration will depend on the amount and frequency of the composition applied and can be adjusted as desired.

Preferably, the transdermal composition has a pH of about 7 to 9. This pH range provides better skin permeation of an anti-Parkinson agent than formulations having a lower pH.

Further, it has been found that the type of the pH-adjusting agent plays an important role on skin permeation and therefore can effectively function as a permeation enhancer. For example, alkanolamine has been found to advantageously provide enhanced skin permeation of anti-Parkinson agents in addition to providing a pH adjustment. Thus, the pH-adjusting agent is preferably selected such that it also functions as a permeation enhancer and the composition exhibits greater transdermal flux of the anti-Parkinson agent than a composition containing an inorganic pH-adjusting agent. Triethanolamine is preferred in particular, especially when the anti-Parkinson agent is pramipexole salt. When triethanolamine is used, permeation enhancement is provided not only from the “mechanical” increase of the apparent pH of the composition, which decreases the ionization rate of the salt form of the drug, but also by a separate mechanism that is not observed when another structurally similar amine compound is used as a permeation enhancer.

The permeation enhancing effect of alkanolamine in the present dosage form takes place in a highly hydrophilic-alcoholic mixture that is free or substantially free of fatty components, where any fatty component is present up to about 1% wt. This is all the more surprising in view of previous studies which show permeation enhancement from the formation of a drug-alkanolamine salt when the drug is solubilized in a highly lipophilic system comprising high amounts of fatty alcohols, fatty acids, or fatty esters.

The permeation enhancing agent can be used in any desired amount. For an alkanolamine permeation enhancer such as triethanolamine, an amount of about 5 to 12% and preferably 7% by weight of the composition is used.

In an embodiment, the invention provides a composition which provides continuous and sustained, steady-state release of an anti-Parkinson agent over a period of time. When the composition provides controlled release over 24 hours, the composition is suitable for once-a-day administration. The composition can be formulated to provide sustained release for a shorter or longer duration by adjusting the amounts of the anti-Parkinson agent and the excipients.

The composition can be administered in any desired amount and frequency. The amount and frequency of dosage will depend on the type and amount of the active agent to be administered and the patient's needs, and can be easily adjusted based on the desired total amount of application, severity of the disease, and efficacy of the drug. As an example, a composition containing an anti-Parkinson agent in an amount of about 0.5 and 5% by weight can be applied to an area of skin of about 50 to 1000 cm² once or several times a day, for a total amount of about 10 grams of the composition. When a smaller dose is desired, the total amount of administration can be reduced accordingly. For example, a total amount of about 4 grams of the composition can be applied to a skin area of about 100 to 400 cm². The end user will appreciate ease and flexibility of application, as the composition can be applied in any desired dosage on any suitable dermal or mucosal surface.

Advantageously, the composition according to the invention can be prepared by simply mixing the anti-Parkinson agent and the inactive excipients to form a homogenous composition. The composition can be packaged in a dosage form for single-dose or multi-dose administration. For example, the composition can be provided in a vacuumed unit dosage container or in a container containing multiple dosages.

### EXAMPLES

The invention is further illustrated in the following examples, which are provided for the purpose of illustration only and do not limit the invention in any way. Although pramipexole is used in the following examples, it will be appreciated that other indolone anti-Parkinson compounds can similarly be used.

In the following examples, evaluation of formulations containing an anti-Parkinson drug was performed using a predictive experimental in vitro permeation model. Pre-clinical in vitro testing of transdermal formulations containing an anti-Parkinson drug was performed using static vertical diffusion cells, which simulates the physiological conditions of in vivo. The model consists of two compartments, donor and receptor, separated by a model skin membrane. The drug formulation is applied onto the skin surface which is maintained at a physiological temperature, and the permeated drug is collected in the receptor compartment containing a physiological receptor medium at regular intervals. Sample HPLC analysis shows a drug kinetic profile, with cumulative absorbed amount as a function of time, as well as a drug flux profile, which is the slope of the former as a function of time, and therefore allows characterization of release properties of the formulations.

The study was performed according the Organisation for Economic Cooperation and Development (OECD) guidance, “Guidance document for the conduct of skin absorption studies” (Mar. 5, 2004). The following conditions were implemented.

1. Diffusion cells: Vertical glass Franz diffusion cells with a receptor compartment of 7.5±0.3 mL and a donor compartment of 3.0 mL and a diffusion area of 1.77 cm² (see Table 1).

TABLE 1
Diffusion Cell Specification

| | |
|---|---|
| Type of cells | Franz vertical |
| Diffusion area | 1.77 cm² |
| Donor compartment | 3 mL |
| volume | |
| Receptor compartment | 7.5 ± 0.3 mL |
| volume | |

2. Receptor solution: Phosphate buffered saline (PBS) at pH 7.4, with addition of 2% w/v Volpo N20 (Oleth-20, oleyl ether of polyoxyethylene glycol), prepared according to SOP QPS-032 [2], maintained at 35° C. during the whole study and stirred at 600 RPM (see Table 2).

TABLE 2
Properties of Receptor Solution

| | |
|---|---|
| Receptor medium | PBS + Volpo |
| | N20 2% |
| Receptor temperature | 35° C. |
| Receptor stirring speed | 600 RPM |

3. Formulation loading: About 10 mg (5.6 mg/cm²) of the formulation was applied with the tip of a plastic syringe plunger and gently spread over the skin diffusion surface. This loading is close to clinical loading, and is consistent with OECD guidelines. Formulations were left in non-occluded conditions, in order to allow the formulations to change as under in-use conditions.

4. Replicates: Each formulation was tested in 4 replicates (3 different donors). Overall, twelve skin samples were used in randomized order over three different donors, one of which was used as internal reference.

5. Excised skin: Fresh pig ear was harvested and processed the same day of permeation (maximum 5 hours delay). The skin samples were sliced, and the thickness of each skin sample was measured with a micrometer. The samples were pre-incubated (for stabilization) for 2 hours on the receptor compartment, in contact with the receptor solution. The porcine skin has been found to have similar morphological and functional characteristics as human skin (see Simon G. A., Maibach H. I., “The pig as an experimental animal model of percutaneous permeation in man: qualitative and quantitative observations,” Skin Pharmacol. Appl. Skin Physiol., 13(5):229-34 (September-October 2000)). In addition, it has been found to permeability characteristics close to that of the human skin (see Andega S., Kanikkannan N., Singh M., “Comparison of the effect of fatty alcohols on the permeation of melatonin between porcine and human skin,” J. Control Release 77(1-2):17-25 (November 2001); Singh S., Zhao K., Singh J., “In vitro permeability and binding of hydrocarbons in pig ear and human abdominal skin,” Drug Chem. Toxicol. 25(1):83-92 (February 2002); Schmook F. P., Meingassner J. G., Billich A., “Comparison of human skin or epidermis models with human and animal skin in in-vitro percutaneous absorption,” Int. J. Pharm. 215(1-2):51-6 (March 2001)). Pig ear skin was preferred over human skin because of its greater supply.

TABLE 3
Properties of Skin Model

| | |
|---|---|
| Species | Pig |
| Gender | Male/Female |
| Age | 5-6 months |
| Region | Ear |
| Origin | Cadaver |
| Condition | Fresh |
| Time harvest/use | Max. 5 h |
| Pre-treatment | None |
| Membrane thickness | Sliced, 1000 ± 200 μm |

6. Skin integrity: Skin integrity was assessed by evaporimetry (TEWL). Skin samples with TEWL>50 g/cm²h were discarded and replaced.

7. Study duration: 24 hours, to correspond to formulations designed to be applied once daily.

8. Sampling frequency: By 4-hour interval, at time points of 8, 12, 16, 20, and 24 hours.

9. Sampling: The studies were performed with a Microette® autosampler (Hanson Research). Receptor solution samples (1.2 mL) were automatically removed at regular interval times (after 0.8 mL receptor compartment priming). Samples were collected in 2 mL HPLC amber glass vials pre-sealed with septum crimp-caps and already containing 10 μL of a solution of 10% trifluoroacetic acid (TFA), for precipitation of macromolecules such as proteins released from the skin (see Table 4).

TABLE 4
Properties of Sampling Performed

| Type of sampling | Automatic |
|---|---|
| Sample volume [mL] | 1.2 |
| Waste volume [mL] | 0.8 |

10. Samples processing: Samples were first transferred into Eppendorf microtubes, and centrifuged at 14500 RPM for 10 minutes. Each supernatant (0.9 mL) was then transferred in a clean 2 mL HPLC amber glass vial and crimp-capped, ready for analysis.

11. Sample analysis: Analysis of the samples was performed by HPLC with UV diode-array detection.

### Example A

### Effect of pH on Pramipexole Hydrochloride Skin Permeation

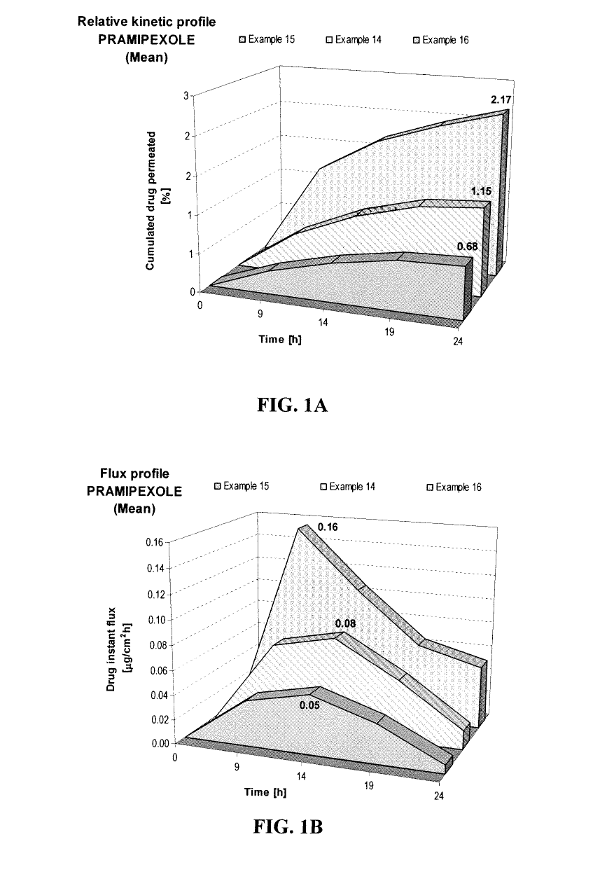

When Examples 1-3 were prepared with the same formulation but different pHs as shown below, the samples showed different skin permeation characteristics. Example 3, at pH 8, was shown to deliver about 1.9 times more pramipexole than Example 2, at pH 6, and about 3.2 times more than Example 1, at pH 4, as shown in FIG. 1A. This comparison shows the importance of pH on the transdermal delivery of pramipexole. Similarly, the maximum instant pramipexole flux was about 2 times higher for Example 3 (pH 8) than for Example 2 (pH 6), and 3.2 times higher than for Example 1 (pH 4), as shown in FIG. 1B.

| | FORMULATION | | |
|---|---|---|---|
| | Example 1 | Example 2 | Example 3 |
| Composition | % w/w | % w/w | % w/w |
| Pramipexole dihydrochloride | 2.00 | 2.00 | 2.00 |
| (as FBE) | | | |
| Permeation enhancing system | 21.00 | 21.00 | 21.00 |
| Hydroxypropyl cellulose | 1.50 | 1.50 | 1.50 |
| (Klucel HF) | | | |
| Ethanol, absolute | 40.00 | 40.00 | 40.00 |
| pH adjusting agent | qs pH 4.0 | qs pH 6.0 | qs pH 8.0 |
| Purified water | qs 100.00 | qs 100.00 | qs 100.00 |

The positive effect of pH is consistent with the ionization theory, and shows that permeation of ionizable active agents, such as pramipexole, is strongly dependent on the pH. It is generally known that net permeability coefficients of acidic and basic drugs will be dictated by the balance between the ionized and unionized drug fractions in direct contact with the surface of the stratum conium (see, for example, S. D. Roy, “Preformulation aspects of transdermal drug delivery systems,” in TRANSDERMAL AND TOPICAL DRUG DELIVERY SYSTEMS 139-166, Ghosh T K, Pfister W R, Yum S I (eds.) (Interpharm Press, Inc., 1997)). The result would depend upon the pKa of the drug and the pH of the formulation, with the unionized fraction readily obtained from the pKa of the drug and the pH of the formulation, as demonstrated in Henderson-Hasselbach equation:

$$pH = pKa + log ( \frac{unionized}{ionized} )$$

Since lipophilic, uncharged species permeate more easily through the stratum conium, the pH of the formulation should represent the best compromise between skin permeability on one hand (with higher pH resulting in better permeation) and skin tolerability on the other (with a formulation displaying a pH ranging between 3 and 10 generally considered as well-tolerated by the skin) (see GHOSH T K, ADEMOLA J, and PFISTER W R, “Transdermal delivery of β-adrenergic therapeutics,” in TRANSDERMAL AND TOPICAL DRUG DELIVERY SYSTEMS 299-325, Ghosh T K, Pfister W R, Yum S I (eds.) (Interpharm Press, Inc., (1997)). For pramipexole, whose pKa is 9.6, ionization is only about 33% at pH of about 10.5 but about 97% at pH of about 8.0, limiting in the latter case the diffusion of the drug through the skin.

### Example B

### Effect of pH on Pramipexole Hydrochloride Skin Permeation

The effect of pH on permeation of pramipexole hydrochloride was studied as in Example A, but at pH of 5 and 8 as shown below.

FORMULATION

| | Example 4 | Example 5 |
|---|---|---|
| Composition | % w/w | % w/w |
| Pramipexole dihydrochloride (as FBE) | 2.00 | 2.00 |
| Permeation enhancing system | 20.00 | 20.00 |
| Hydroxypropyl cellulose (Klucel HF) | 1.50 | 1.50 |
| Ethanol, absolute | 40.00 | 40.00 |
| pH adjusting agent | qs pH 5.0 | qs pH 8.0 |
| Purified water | qs 100.00 | qs 100.00 |

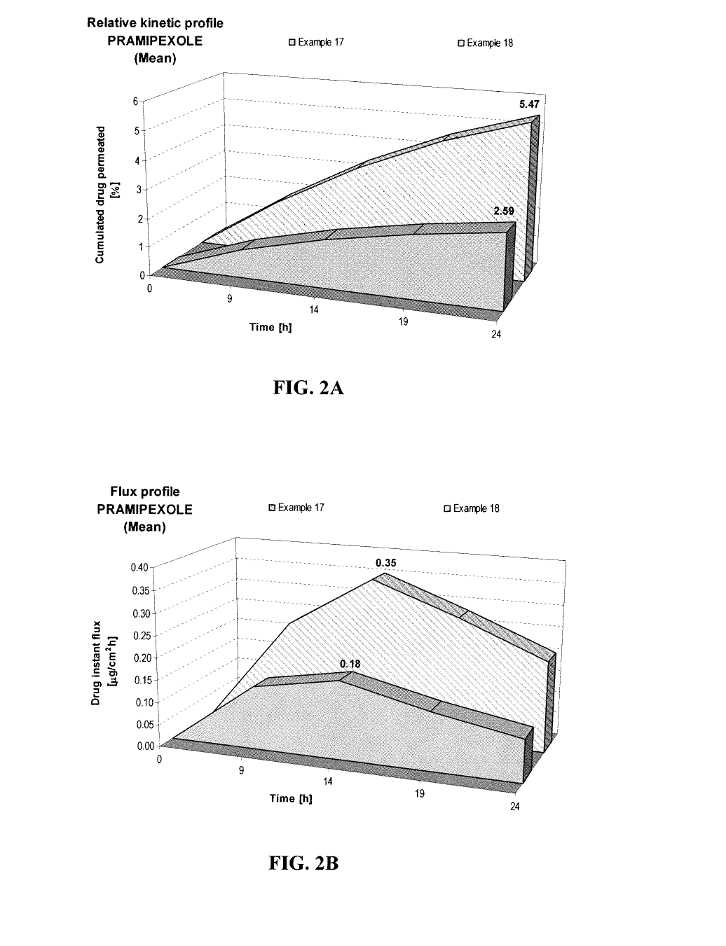

Increasing the pH from 5 (Example 4) to pH 8 (Example 5) led to a 2.1-fold increase of 24-hour cumulated Pramipexole amount, as shown in FIG. 2A. The maximum instant pramipexole flux also increased by about 100%, as shown in FIG. 2B. This study further shows the benefit of increasing pH for pramipexole skin permeation.

### Example C

### Effect of pH-Adjusting Agent on Pramipexole Hydrochloride Skin Permeation

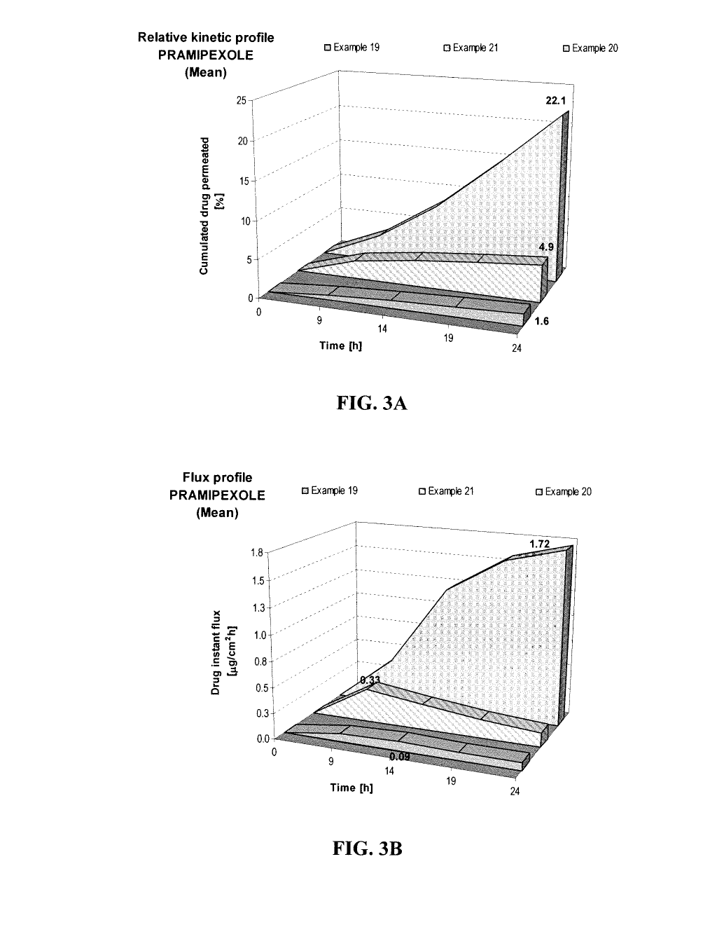

This study shows that, surprisingly, the type of the pH-adjusting agent also plays an important role on pramipexole skin permeation. For example, adjusting the pH of a pramipexole formulation with triethanolamine (Example 7) resulted in about 4.5-fold higher delivery of pramipexole than when diisopropylamine, which is structurally very similar to triethanolamine, is used as the pH-adjusting agent (Example 8) and about 13.8-fold higher delivery of pramipexole than when sodium hydroxide is used as the pH-adjusting agent (Example 6). The maximum pramipexole instant flux showed the same pattern, with the measurement for Example 7 being about 5.2-fold higher than Example 8, and about 19-fold higher than Example 6. The results are graphically shown in FIGS. 3A-3B.

| | FORMULATION | | |
|---|---|---|---|
| | Example 6 | Example 7 | Example 8 |
| Composition | % w/w | % w/w | % w/w |
| Pramipexole dihydrochloride | 2.00 | 2.00 | 2.00 |
| (as FBE) | | | |
| Permeation enhancing system | 21.00 | 21.00 | 21.00 |
| Hydroxypropyl cellulose | 1.50 | 1.50 | 1.50 |
| (Klucel HF) | | | |
| Ethanol, absolute | 40.00 | 40.00 | 40.00 |
| Sodium hydroxide 1M | qs pH 8.0 | | |
| Triethanolamine | | qs pH 8.0 | |
| Diisopropylamine | | | qs pH 8.0 |
| Purified water | qs 100.00 | qs 100.00 | qs 100.00 |

### Example D

### Effect of Triethanolamine Concentration on Pramipexole Hydrochloride Skin Permeation

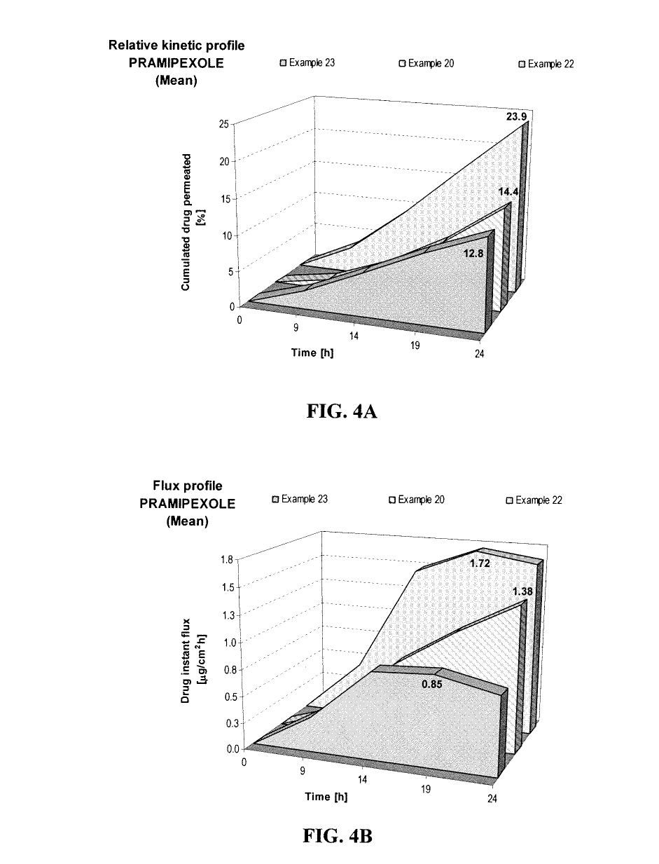

This study showed that, surprisingly, increasing the amount of triethanolamine, used as a pH-adjusting agent, from 5% wt (Example 11) to 7% (Example 10), and then to 8.85% (Example 9) does not necessary result in an increase of pramipexole skin permeation, as graphically illustrated in FIGS. 4A and 4B. On the contrary, the highest transdermal delivery of pramipexole was achieved when triethanolamine was provided in the amount of 7%, while the lower triethanolamine amount of 5% and the highest triethanolamine amount of 8.85% resulted in similar pramipexole permeation levels, at about half of that in Example 10. The highest maximum instant pramipexole flux was also achieved with the triethanolamine amount of 7%. Although the lowest maximum instant flux (0.85 mcg/cm²·h) was obtained with the lowest triethanolamine amount of 5%, increasing the amount of triethanolamine to 8.85% did not result in a superior flux value than in Example 10, and also did not even reached the maximum instant flux within 24 hours. These results were completely unexpected because triethanolamine is an amine compound with strong alkalinizing properties and increasing triethanolamine increases the pH. This study shows that there is actually an optimum concentration of triethanolamine in a pramipexole formulation, beyond which skin permeation of pramipexole is not improved and may even be impaired.

| | FORMULATION | | |
|---|---|---|---|
| | Example 9 | Example 10 | Example 11 |
| Composition | % w/w | % w/w | % w/w |
| Pramipexole dihydrochloride | 2.00 | 2.00 | 2.00 |
| (as FBE) | | | |
| Permeation enhancing system | 21.00 | 21.00 | 21.00 |
| Hydroxypropyl cellulose | 1.50 | 1.50 | 1.50 |
| (Klucel HF) | | | |
| Ethanol, absolute | 40.00 | 40.00 | 40.00 |
| Triethanolamine | 8.85 | 7.00 | 5.00 |
| Purified water | qs 100.00 | qs 100.00 | qs 100.00 |

### Example E

### Skin Permeation of Pramipexole Salt Versus Pramipexole Free Base

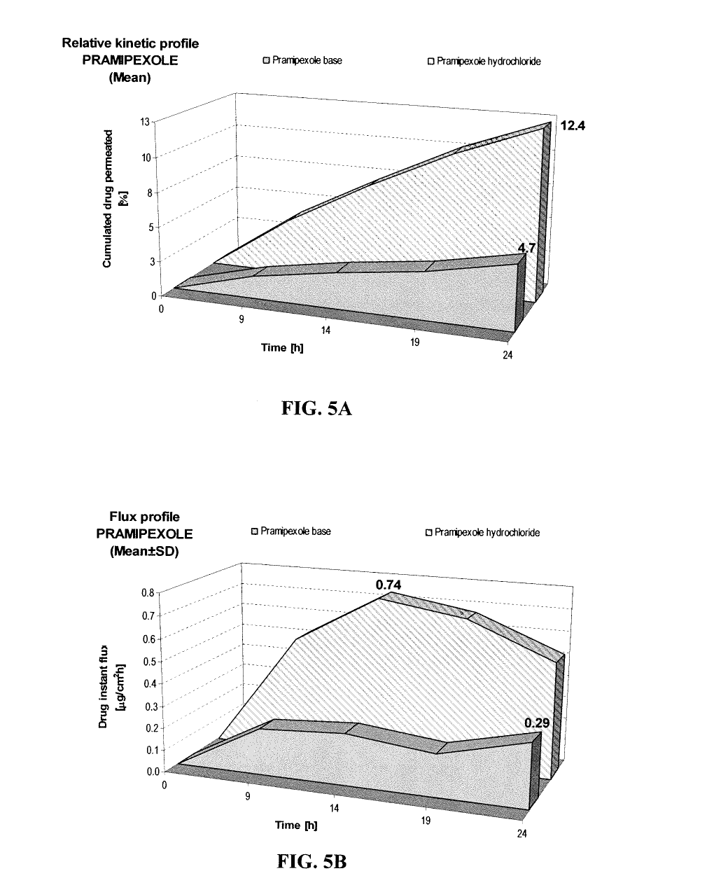

This study showed that, at a given pH (i.e., at identical ionization rate), the permeation of the free base form of pramipexole is surprising lower than that of the hydrochloride salt form. Indeed, the cumulated amount of pramipexole permeated over 24 hours was about 2.6 times higher in the example containing the salt (Example 12) than the example containing the free base (Example 13). Similarly, the maximum instant flux of pramipexole was also 2.6 higher for the hydrochloride salt (Example 12) than for the free base (Example 13). Triethanolamine was present in both formulations, although in a lower concentration in Example 13 than in Example 12 in order not to artifact the interpretation of data with different pH. This study shows that triethanolamine provides better permeation enhancement results when used in combination with the hydrochloride form of pramipexole than the free base form of pramipexole. The results are graphically illustrated in FIGS. 5A-5B.

| | FORMULATION | |
|---|---|---|
| | Example 12 | Example 13 |
| Composition | % w/w | % w/w |
| Pramipexole dihydrochloride | 2.00 | — |
| (as FBE) | | |
| Pramipexole free base | — | 2.00 |
| Permeation enhancing system | 26.00 | 26.00 |
| Antioxidant | 0.40 | 0.40 |
| Hydroxypropyl cellulose | 1.50 | 1.50 |
| (Klucel HF) | | |
| Ethanol, absolute | 40.00 | 40.00 |
| Triethanolamine | qs pH 7.0 +/− 0.1 | — |
| Triethanolamine | — | qs pH 7.0 +/− 0.1 |
| Purified water | qs 100.00 | qs 100.00 |

Examples A to E demonstrate that triethanolamine enhances skin permeation of pramipexole not only because of a “mechanical” increase of the apparent pH of the formulation, and therefore a decrease of the ionization rate of pramipexole salt such as pramipexole hydrochloride, but also by a different and specific mechanism that is not observed with other amine compounds that are structurally similar to triethanolamine. Hence, triethanolamine surprisingly acts as a true permeation enhancer for pramipexole salt, in addition to its function as a pH-adjusting agent. The examples further demonstrate that the permeation enhancing effect of triethanolamine observed in formulations according to the invention takes place in a highly hydrophilic-alcoholic mixture, free or substantially free of fatty components (typically about 1% wt). This is all the more surprising in view of the previous studies showing the effect of drug-alkanolamine salt formation when the drug is solubilized in highly lipophilic system, comprising high amounts of fatty alcohols, fatty acids, or fatty esters.

### Example F

### Effect of pH-Adjusting Agent on Nicotine Hydrogenotartrate Skin Permeation

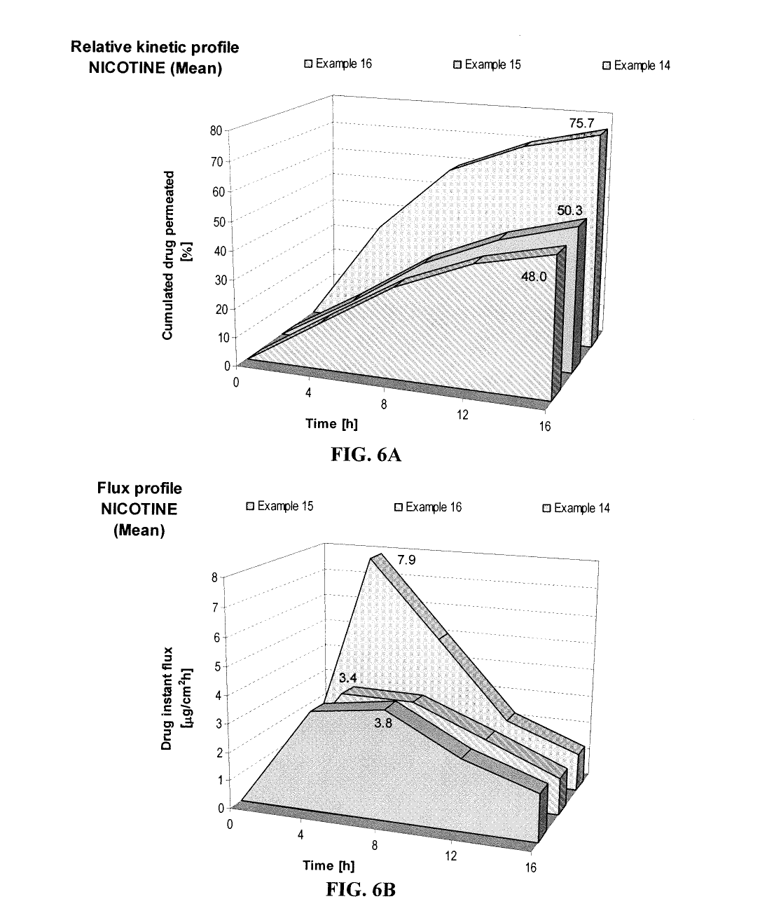

This study shows that, surprisingly, the type of the pH-adjusting agent plays an important role on nicotine skin permeation which is opposite to the findings made on pramipexole in Example C. For example, adjusting the pH of a nicotine formulation with diethanolamine (Example 14) resulted in about 55 percent higher delivery of nicotine than when triethanolamine (Example 15) or diisopropylamine (Example 16), which are organic amines structurally very similar to diethanolamine, are used as the pH-adjusting agent. The maximum nicotine instant flux showed the same pattern, with the measurement for Example 14 being about 2.2-fold higher than Example 15 and Example 16. The results are graphically shown in FIGS. 6A-6B.

| | FORMULATION | | |
|---|---|---|---|
| | Example 14 | Example 15 | Example 16 |
| Composition | % w/w | % w/w | % w/w |
| Nicotine di-H tartrate 2H2O | 4.60 | 4.60 | 4.60 |
| Permeation enhancing system | 20.00 | 20.00 | 20.00 |
| Hydroxypropyl cellulose | 1.50 | 1.50 | 1.50 |
| (Klucel HF) | | | |
| Ethanol, absolute | 40.00 | 40.00 | 40.00 |
| Diethanolamine | 3.55 | — | — |
| Triethanolamine | — | 3.55 | — |
| Diisopropylamine | — | — | 3.55 |
| Purified water | qs 100.00 | qs 100.00 | qs 100.00 |

### Example G

### Effect of pH-Adjusting Agent on Pramipexole Hydrochloride Skin Permeation

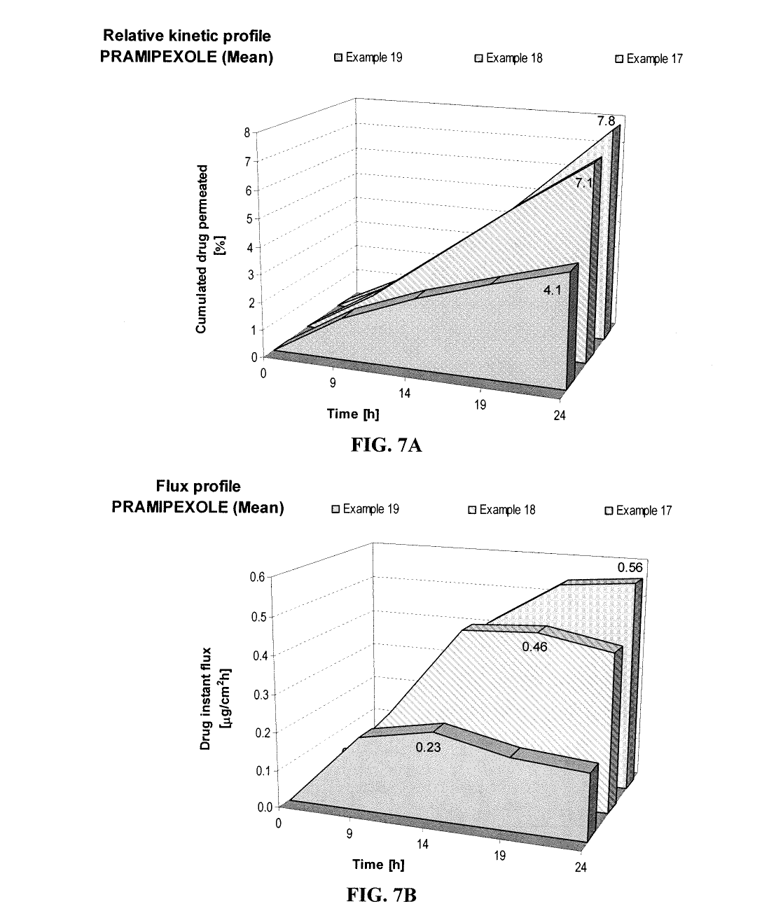

This study confirms that, surprisingly, the type of the pH-adjusting agent plays an important role on pramipexole skin permeation. For example, adjusting the pH to about 7.4+/−0.2 of a pramipexole formulation with triethanolamine (Example 17) resulted in about 1.9-fold higher delivery of pramipexole than when meglumine (Example 19), which is an organic amine structurally very similar to triethanolamine, is used as the pH-adjusting agent. Noteworthy, meglumine concentration in Example 19 (about 2.3% wt) was selected so that pH is the same than in Example 17 (7.4+/−0.2). The maximum pramipexole instant flux showed the same pattern, with the measurement for Example 17 being about 2.4-fold higher than Example 19. Noteworthy, further increasing meglumine concentration up to 4.0% (Example 18), i.e. the same concentration as triethanolamine in Example 17, did allow increasing both the 24-hour cumulated permeated pramipexole and the pramipexole maximal instant flux. However, the 24-hour cumulated permeated pramipexole at 4.0% meglumine (Example 18) is not superior to those of Example 17, despite the higher pH (9.0+/−0.2 versus 7.4+/−0.2, respectively). Strikingly, the pramipexole maximal instant flux is even lower than in Example 17 (0.46 mg/cm²h versus 0.56 mg/cm²h, respectively). The results are graphically shown in FIGS. 7A-7B.

| | FORMULATION | | |
|---|---|---|---|
| | Example 17 | Example 18 | Example 19 |
| Composition | % w/w | % w/w | % w/w |
| Pramipexole dihydrochloride | 2.00 | 2.00 | 2.00 |
| (as FBE) | | | |
| Permeation enhancing system | 21.00 | 21.00 | 21.00 |
| Hydroxypropyl cellulose | 1.50 | 1.50 | 1.50 |
| (Klucel HF) | | | |
| Ethanol, absolute | 40.00 | 40.00 | 40.00 |
| Triethanolamine | 4.00 | — | — |
| Meglumine | — | 4.00 | 2.27 |
| Purified water | qs 100.00 | qs 100.00 | qs 100.00 |

### Example H

### Effect of pH-Adjusting Agent on Pramipexole Hydrochloride Skin Permeation

This study confirms that, surprisingly, the type of the pH-adjusting agent plays an important role on pramipexole skin permeation. For example, adjusting the pH to about 7.4+/−0.2 of a pramipexole formulation with triethanolamine (Example 17) resulted in about 2-fold higher delivery of pramipexole than when diethanolamine (Example 21), which is an organic amine structurally very similar to triethanolamine, is used as the pH-adjusting agent. Noteworthy, diethanolamine concentration in Example 21 (about 1.3% wt) was selected so that pH is the same than in Example 17 (7.4+/−0.2). The maximum pramipexole instant flux showed the same pattern, with the measurement for Example 17 being about 2.4-fold higher than Example 21 (1.34 mg/cm²h versus 0.67 mg/cm²h, respectively).

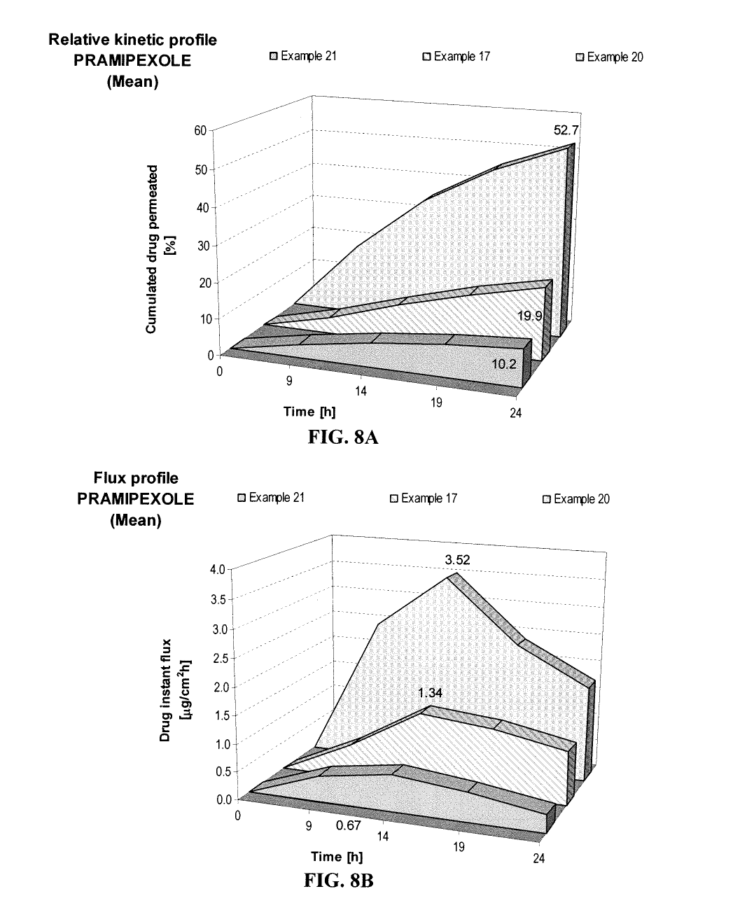

Noteworthy, further increasing diethanolamine concentration up to 4.0% (Example 20), i.e. the same concentration as triethanolamine in Example 17, did allow increasing both the 24-hour cumulated permeated pramipexole and the pramipexole maximal instant flux. Both the 24-hour cumulated permeated pramipexole and the pramipexole maximal instant flux at 4.0% meglumine (Example 20) are markedly superior (about 2.6 times higher) to those of Example 17. This is, however, obtained in detriment of skin tolerance and local irritation, as the high diethanolamine concentration is responsible for a significantly higher pH (9.0+/−0.2 versus 7.4+/−0.2, respectively) far from physiological pH of the skin. The results are graphically shown in FIGS. 8A-8B.

| | FORMULATION | | |
|---|---|---|---|
| | Example 17 | Example 20 | Example 21 |
| Composition | % w/w | % w/w | % w/w |
| Pramipexole dihydrochloride | 2.00 | 2.00 | 2.00 |
| (as FBE) | | | |
| Permeation enhancing system | 21.00 | 21.00 | 21.00 |
| Hydroxypropyl cellulose | 1.50 | 1.50 | 1.50 |
| (Klucel HF) | | | |
| Ethanol, absolute | 40.00 | 40.00 | 40.00 |
| Triethanolamine | 4.00 | — | — |
| Diethanolamine | — | 4.00 | 1.30 |
| Purified water | qs 100.00 | qs 100.00 | qs 100.00 |

What is claimed is:

1. A transdermal dosage form comprising:
a therapeutically effective amount of pramipexole or a pharmaceutically acceptable salt thereof;
a carrier comprising a mixture of water and at least one short-chain alcohol; and
at least one primary permeation enhancer comprising an alkanolamine in an amount sufficient to increase permeation through dermal or mucosal surfaces compared to formulations where the alkanolamine is not utilized,
wherein the apparent pH of the dosage form is between about 7 and 9.

2. The dosage form of claim 1, wherein pramipexole is provided as a free base pramipexole.

3. The dosage form of claim 1, wherein pramipexole is provided as a pharmaceutically acceptable salt of pramipexole.

4. The dosage form of claim 3, wherein the pharmaceutically acceptable salt is pramipexole hydrochloride or dihydrochloride.

5. The dosage form of claim 1, wherein pramipexole is present at a concentration of about 0.5 to about 5 weight percent expressed as free base equivalent.

6. The dosage form of claim 1, wherein the short-chain alcohol is selected from the group consisting of ethanol, propanol, isopropanol, and mixtures thereof and is present in an amount of about 30 to 80% by weight of the dosage form.

7. The dosage form of claim 1, wherein the alkanolamine is selected from the group consisting of monoethanolamine, diethanolamine, triethanolamine, diisopropylamine, meglumine, mixtures thereof, and derivatives thereof.

8. The dosage form of claim 7, wherein the preferred alkanolamine is diethanolamine, triethanolamine, mixtures thereof, and derivatives thereof.

9. The dosage form of claim 1, wherein the carrier further comprises a non-volatile solvent.

10. The dosage form of claim 1, wherein the carrier further comprises an antioxidant.

11. The dosage form of claim 1, wherein the carrier further comprises a thickening agent.

12. The dosage form of claim 1, wherein the carrier further comprises a secondary permeation enhancer.

13. The dosage form of claim 1, wherein the therapeutically effective amount of pramipexole or a pharmaceutically acceptable salt thereof is between about 0.5 to about 5 weight percent; the carrier further comprises a non-volatile solvent, a secondary permeation enhancer, an antioxidant, and a thickening agent; and the primary permeation enhancer comprises triethanolamine.

14. The dosage form of claim 1, wherein the transdermal flux of pramipexole in the dosage form is greater than the transdermal flux of an equal concentration of pramipexole in a composition comprising an inorganic pH-adjusting agent and having a substantially identical pH.

15. A method for producing a dosage form of claim 1, which method comprises mixing a therapeutically effective amount of pramipexole or a pharmaceutically acceptable salt thereof; a carrier comprising water, at least one short-chain alcohol, and at least one thickening agent; at least one antioxidant; and at least one alkanolamine to form a homogeneous composition, wherein the pH of the dosage form is between about 7 and 9.

16. The method of claim 15, which further comprises providing the composition in a substantially airless unidose or multidose dispensing container.

17. A method for administering pramipexole to a human subject in need thereof, which method comprises:
providing a transdermal dosage form comprising a therapeutically effective amount of pramipexole or a pharmaceutically acceptable salt thereof; a hydroalcoholic carrier comprising a thickening agent; at least one antioxidant; and at least one primary permeation enhancer comprising an alkanolamine in an amount sufficient to increase permeation through dermal or mucosal surfaces compared to formulations where the alkanolamine is not utilized, wherein the pH of the dosage form is between about 7 and 9; and
applying the dosage form onto an area of skin of the subject in an amount sufficient to provide a therapeutic concentration of pramipexole in the bloodstream of the subject.

18. The method according to claim 17, wherein the human subject is in need of pramipexole to treat a neurological disorder.

19. The method according to claim 18, wherein the human subject is in need of pramipexole to treat a condition selected from the group consisting of Parkinson's Disease, Restless Legs Syndrome, Tourette's Syndrome, Chronic Tic Disorder, Essential Tremor, and Attention Deficit Hyperactivity Disorder.

20. The method according to claim 17, wherein the amount of pramipexole expressed as free base equivalent in the dosage form is about from 0.5 to about 5 weight percent and the method comprises applying up to about 10 grams of the dosage form daily to a skin surface area of about 50 to about 1500 cm².

21. The method according to claim 17, wherein the amount of pramipexole expressed as free base equivalent in the dosage form is about from 1.5 to about 3 weight percent and the method comprises applying up to about 5 grams of the dosage form daily to a skin surface area of between about 100 to about 800 cm².

22. The method according to claim 17, wherein the method comprises applying the dosage form dose in a single dose or multiple doses daily.

23. A dosage form comprising pramipexole or a pharmaceutically acceptable salt thereof, wherein the dosage form provides sustained, steady-state delivery of pramipexole for about 24 hours.

24. The dosage form of claim 22, wherein pramipexole is provided in an amount of from about 0.5 to 5 weight percent and the dosage form is a non-occlusive composition for transdermal delivery of pramipexole.

25. A method for treating a movement disorder in a subject in need thereof by administering the dosage form of claim 23 to the subject.

26. Use of at least one primary permeation enhancer comprising an alkanolamine in a transdermal dosage form that includes a therapeutically effective amount of pramipexole or a pharmaceutically acceptable salt thereof and a carrier comprising a mixture of water and at least one short-chain alcohol and that has an apparent pH of between about 7 and 9; wherein the alkanolamine is present in the dosage form in an amount sufficient to increase permeation through dermal or mucosal surfaces compared to formulations where the alkanolamine is not utilized.
